(19) United States
(12) Patent Application Publication
(10) Pub. No.: US 2023/0263997 A1
(43) Pub. Date: Aug. 24, 2023

(54) GUIDING ASSEMBLY FOR CATHETERS

(71) Applicant: OZYEGIN UNIVERSITESI, Istanbul (TR)

(72) Inventors: Guney Guven YAPICI, Istanbul (TR); Ali VAHIDYEGANEH, Istanbul (TR)

(73) Assignee: OZYEGIN UNIVERSITESI, Istanbul (TR)

(21) Appl. No.: 17/923,627

(22) PCT Filed: May 7, 2020
(86) PCT No.: PCT/TR2020/050399

§ 371 Date: Nov. 7, 2022

(51) Int. Cl. A61M 25/01

(52) U.S. Cl. CPC A61M 25/0158; A61M 2205/0266

(57) ABSTRACT

A guiding assembly for catheters comprises two coupling means; a shape memory alloy (SMA) actuator which is electrically actuatable by providing resistive heating resulting from an electric current, wherein the SMA actuator is fixed to the two coupling means and at least one part of the SMA actuator is positioned between the two coupling means; a super-elastic alloy (SEA) member fixed to the two coupling means, wherein at least one part of the SEA member is positioned between the two coupling means. The guiding assembly may further comprise at least one deformation sensor for measuring deformation levels of the SMA actuator.

### CROSS REFERENCES TO THE RELATED APPLICATION

The application is the national phase entry of International Application No. PCT/TR2020/050399, filed on May 7, 2020, the entire content of which is incorporated herein by reference.

### TECHNICAL FIELD

The present invention relates a guiding assembly for catheters used in medical diagnosis or treatment, especially in minimal invasive surgical procedures.

### BACKGROUND

Catheters are medical devices in a form of a tube that may be inserted into a body lumen such as vessel, cavity or duct. Typically, catheters are relatively thin and flexible to facilitate advancement/retraction along non-linear paths of the lumen. Catheters may be employed for a wide variety of purposes, including the internal bodily positioning of diagnostic and/or therapeutic devices. For example, catheters may be employed to position internal imaging devices (e.g., ultrasound transducers), to deploy implantable devices (e.g., stents, stent grafts, vena cava filters), and/or to deliver therapy (e.g., ablation catheters, drug delivery).

During the advancement and retraction of the catheter in the body lumen, catheter tip such as nelaton tip must be guided through the body lumen. In the state of the art, catheters are directed by a guide wire passing through the catheter. The guide wire is operated from an open end extending out of the body. The operation of the guide wire must be exercised by an experienced operator because the operation depends on tactile and dexterity capability of the operator for guiding catheter tip. Since there is no feedback mechanism providing information about shape of the catheter such as bending degree of the catheter tip, operators have a difficulty to correctly direct the catheter tip which may traumatize the body lumen. Another problem in the state of the art arises from the guide wire which is able to bend only in one direction. Although it is possible to bend the catheter tip in 360° by spinning the wire, spinning catheter may also traumatize the body lumen. Yet another problem in the state of the art is limited bending capability of the guide wires. For high branch angle in the body lumen, especially above 100°, it is almost impossible to bend the catheter tip by conventional guiding apparatus. Yet another problem in the state of the art is long transition period between bent position and initial position (generally a straight position) of the catheter tip. The long transition period prolongs advancement and retraction of the catheter which increases the infection risks. On the other hand, the initial straight position cannot be maintained anymore after a few cycles of the bent position to initial position transition.

In the state of the art, EP2629674 discloses a catheter with shape memory alloy actuator that includes at least a first shape memory member that is actuatable to affect at least a portion of the oscillating movement of the load. The actuator may further include a second shape memory member actuatable to affect at least a second portion of the oscillating movement of the load.

However, there is still a need in the art for catheters for medical diagnosis or treatment, especially in minimal invasive surgical procedures, which can be guided precisely, correctly and reliably in the body.

### BRIEF DESCRIPTION OF THE DRAWINGS

An exemplary embodiment of the present invention is illustrated by way of example in the accompanying drawings to be more easily understood and uses thereof will be clearer when considered in view of the detailed description, in which like reference numbers indicate the same or similar elements, and the following figures in which:

FIG. 1 is a straight view of a guiding assembly mounted on a catheter tube in one exemplary embodiment of the present invention wherein SMA actuator fixed to the coupling means.

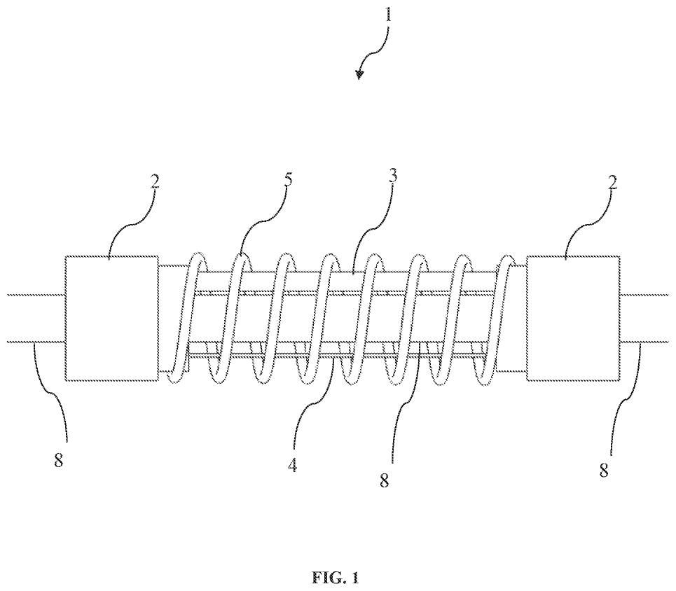

FIG. 2 is a bent view of guiding assembly mounted on a catheter tube in one exemplary embodiment of the present invention wherein SMA actuator fixed to the coupling means.

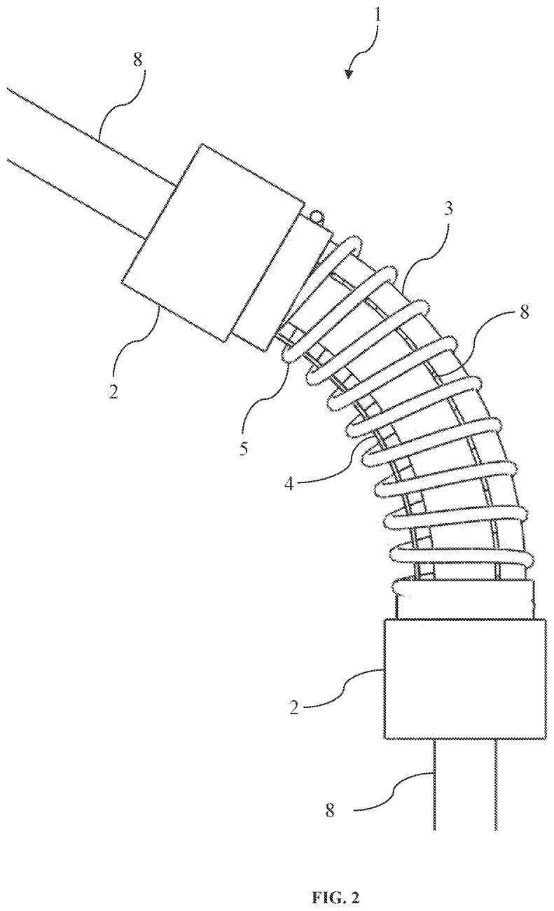

FIG. 3 is a view of the coupling showing openings in one exemplary embodiment of the present invention.

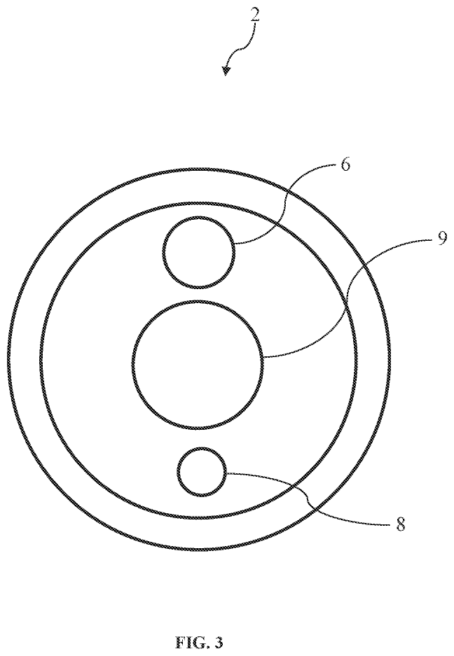

FIG. 4 is a perspective view of the resilient member in the form of a helical spring covering catheter tube, the SMA actuator and the SEA member in one exemplary embodiment of the present invention.

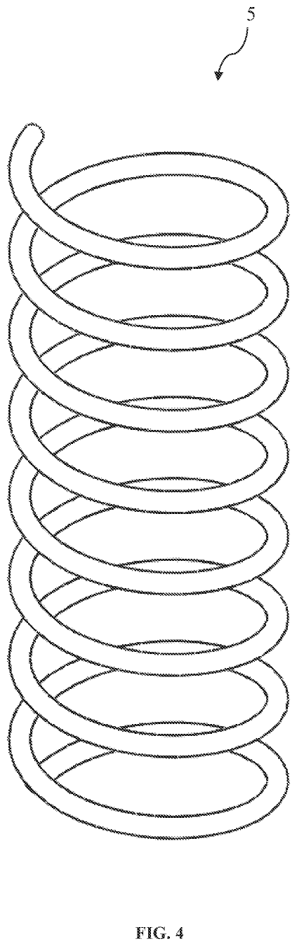

The elements illustrated in the figures are numbered as follows:
1. Guiding assembly
2. Coupling means
3. Shape memory alloy (SMA) actuator
4. Super-elastic alloy (SEA) member
5. Resilient member
6. SMA actuator opening
7. Catheter tube
8. SEA member opening
9. Catheter tube opening

### DETAILED DESCRIPTION OF THE EMBODIMENTS

Embodiments of the present invention relates to a guiding assembly (1) for catheters comprising
at least two coupling means (2);
at least one shape memory alloy (SMA) actuator (3) which is electrically actuatable by providing resistive heating resulting from an electric current wherein SMA actuator (3) fixed to the coupling means (2) and at least one part of SMA actuator (3) is positioned between coupling means (2);
at least one super-elastic alloy (SEA) member (4) which is fixed to the coupling means (2) or SMA actuator (3) wherein at least one part of SEA member (4) is positioned between coupling means (2).

The SMA actuator (3) has an initial shape at or below a transition start temperature T_{S} and a final shape at or above a transition finish temperature T_{F} and has transitional shapes between the initial shape and the final shape which are formed according to the temperature between the transition start temperature T_{S} and the transition finish temperature T_{F} of the shape memory alloy layer (2).

Recovery force of the SEA member (4) positions the SMA actuator (3) into initial shape from transitional shapes or final shape when the electric current is cut.

When an electric current is applied to the SMA actuator (3), the current passes through the SMA actuator (3) and the SMA actuator (3) starts to get heated by resulting resistive heating. When temperature of the SMA actuator (3) reaches the transition start temperature T_{S}, the SMA actuator (3) starts to deform to the final shape from the initial shape. In a temperature between the transition start temperature T_{S} and the transition finish temperature T_{F}, the SMA actuator (3) is deformed to a transitional shape. If the temperature reaches at or above the transition finish temperature T_{F}, form of the SMA actuator (3) is in the final shape. During this, the SEA member (4) acts against the deformation of the SMA. The SEA member (4) exerts an opposing force for movement of the SMA actuator (3) from the initial shape to a transitional shape or from a transitional shape to the final shape.

As used herein, the transition temperature also means the transformation temperature.

When the applied current is cut, the SMA actuator (3) starts to cool down and to deform back to the initial shape from the final shape or a transitional shape, if the SMA has a two-way memory effect. If the temperature reaches below to the transition start temperature T_{S}, form of the SMA actuator (3) is in the initial shape. Since the SMA actuator (3) is not actively cooled, the duration for cooling and thus deforming back to the initial shape of the SMA actuator (3) is relatively high for guiding the attached catheter. For reducing the duration of these reverse deformations such as from a bent form/shape to a straight form/shape of the catheter, the SEA member (4) acts in favor of the deformations. The SEA member (4) exerts a recovery force for the reverse deformations of the SMA actuator (3) from the final shape to the transitional shape or from a transitional shape to the initial shape. By this way, duration for an actuation cycle is significantly reduced.

Moreover, thanks to the SEA member (4), the SMA actuator (3) which has one-way memory effect may also be used for the guiding assembly (1). An SMA actuator (3) with one-way memory effect is deformed when temperature exceeds the transition start temperature T_{S}. Then, when the temperature decreases below the transition start temperature T_{S}, the SMA actuator (3) keeps its deformed shape such as the final shape and cannot deform back to the initial shape by itself. However, via the recovery force exerted by the SEA member (4), the SMA actuator (3) is deformed to the initial shape.

In one embodiment of the present invention, at least one deformation sensor provided along at least a part of the SEA member (4) for measuring deformation (change in length per original length) of the SEA member (4) indicating current shape of the guiding assembly (1). Since the strain value of the SEA member (4) and thus of the SMA actuator (3) are directly related to shape of the SMA actuator (3), each strain value corresponds to a specific shape of the SMA actuator (3). Thus, the current shape (the initial shape, the final shape or any transitional shape) of the SMA actuator (3) which is also shape/form of the guiding assembly (1) is determined by tracking the strain value from the deformation sensor. The strain value can be used as a feedback to a system to control the guiding assembly.

The SEA member (4) provides a perfect substrate for deformation sensor. The SEA member (4) is pseudoelastic, and thus does not retain permanent deformation during motions (transformations) of the SMA actuator (3). The SMA actuator (3) deforms back into exact initial shape with the help of the recovery force applied by the SEA member (4). Any other elastic material other than SEA member (4) may position the SMA actuator (3) to the initial shape with a residual deformation (slight, but important, difference according to the initial shape) after a few cycles/actuations. The residual deformation on the SMA actuator (3) may lead to an erroneous “strain value/shape of guiding assembly (1)” calibration error causing faulty determination of current shape of the guiding assembly (1) which makes the operator to excessively or deficiently guide the catheter.

In one embodiment of the present invention, at least one deformation sensor is preferably a resistance based and/or thin film/foil based, and/or semiconductor based and/or piezo based (piezoresistive or piezoelectric) and/or fiber bragg grating based strain gauge.

In one embodiment of the present invention, a deformation sensor (4) which is susceptible for thermal drifting may be used. For this kind of embodiments, any temperature compensation method or means such as using dummy gauge technique with Wheatstone bridge, covering the SMA actuator (3)/SEA member (4) with an insulating coat may be provided.

In one embodiment of the present invention, the deformation sensor is provided along at least a part of the SEA member (4) by mechanical or metallurgical fastening or chemicals such as adhesive (for instance cyanoacrylates or epoxies) means or method. In preferred embodiment of the invention, the deformation sensor is provided by a thermally insulative and biocompatible adhesive for minimizing thermal drifting on the deformation sensor. The adhesive may also or solely be epoxy based and temperature resistant to preferably above 60° C.

In one embodiment of the present invention, the guiding assembly (1) comprises a thermal insulator for thermally insulating the SEA member (4).

In one embodiment of the present invention, the guiding assembly (1) comprises a first terminal connected to deformation sensor for receiving signal for strain values.

In one embodiment of the present invention, the guiding assembly (1) comprises a second terminal connected to the SMA actuator (3) for providing electric current to the SMA actuator (3).

In one embodiment of the present invention, the SMA actuator (3) and/or the SEA member (4) are preferably a NiTi alloy and/or copper-based alloys such as, but not limited to, CuZnAl, CuMnAl, CuZnNi and/or iron-based such as FeMnSi, FeMnAl, FePt, cobalt-based and/or titanium-based (without nickel). In one embodiment of the present invention, the SMA actuator (3) is a NiTi alloy with nominal composition of 54.5% Nickel and 45.5% Titanium which has shape memory properties and transition temperatures is between 30° C. (transition start temperature T_{S})-60° C. (transition finish temperature T_{F}). The SEA member (4) is a NiTi alloy with nominal composition of 56% Nickel and 44% Titanium having superelasticity (pseudoelasticity) properties above 10° C.

In one embodiment of the present invention, SMA actuator (3) and/or the SEA member (4) are in the form of wire.

In one embodiment of the present invention, initial shape is in a straight form and the final shape is in a bent form or initial shape is in a bent form and the final shape is in a straight form. In a variation of this embodiment bent form may be up to 180° C. (C shape) form.

In one embodiment of the present invention, the guiding assembly (1) further comprises a resilient member (5) for assisting to position the SMA actuator (3) into initial shape from transitional shapes or final shape when the electric current is cut. In one alternative of this embodiment, the resilient member (5) is in the form of a helical spring. In another alternative of this embodiment, the helical spring is covering at least the SMA actuator (3) and the SEA member (4).

In one embodiment of the present invention, SMA actuator (3) and the SEA member (4) extend longitudinally between coupling means (2).

In one embodiment of the present invention, the coupling means (2) comprises SMA actuator opening (6) and SEA member opening (8) wherein the terminals of SMA actuator and SEA member can fit respectively. In addition, the coupling means (2) comprises catheter tube opening (9) for receiving a catheter (7).

In one embodiment of the present invention, the coupling means (2) comprises recesses for mounting each SMA actuator (3) and/or SEA member (4) and/or resilient member (5). The coupling is to be of biocompatible and electrically insulative material.

In one embodiment of the present invention, the guiding assembly (1) comprises a fastening means such as, but not limited to, screws, bolts and rivets for fastening a catheter to the coupling means (2).

In one embodiment of the present invention, the guiding assembly (1) comprises an external cover or coating surrounding at least the SMA actuator (3) and the SEA member (4) for providing smoothness while advancement or retraction of the catheter along non-linear paths of the lumen and for providing any frictional or heat based damage to the epitel. The cover shall be of biocompatible material.

In one embodiment of the present invention, the SEA member (4) has a transition temperature interval different, preferably lower, than an interval between the transition start temperature T_{S} and the transition finish temperature T_{F}. Thus, shape/form and mechanical properties of the SEA member (4) is perfectly unaffected by the stress induced during the motion of the SMA actuator (3). SEA member (4) shall also be perfectly unaffected by the raised temperature of the SMA actuator (3).

In another embodiment, the present invention provides a catheter comprising the guiding assembly of the present invention. Accordingly, the guiding assembly (1) can be a part of the catheter (7).

1. A guiding assembly for catheters, comprising:
at least two coupling means;
at least one shape memory alloy (SMA) actuator electrically activated by providing resistive heating resulting from an electric current, wherein the at least one SMA actuator is fixed to the at least two coupling means and at least one part of the at least one SMA actuator is positioned between the at least two coupling means; and
at least one super-elastic alloy (SEA) member fixed to the at least two coupling means or the at least one SMA actuator, wherein at least one part of the at least one SEA member is positioned between the at least two coupling means;
wherein the at least one SMA actuator has an initial shape below a transition start temperature T_{S}, a final shape at or above a transition finish temperature TF, and transitional shapes between the initial shape and the final shape according to the temperature between the transition start temperature T_{S} and the transition finish temperature TF of the at least one SMA actuator, wherein recovery force of the at least one SEA member positions the at least one SMA actuator into the initial shape from the transitional shapes or the final shape when the electric current is cut.

2. The guiding assembly for catheters according to claim 1, wherein at least one deformation sensor provided along at least a part of the at least one SMA actuator or of the at least one SEA member for measuring deformation levels of the at least one SMA actuator or the at least one SEA member indicating a current shape of the guiding assembly.

3. The guiding assembly for catheters according to claim 2, wherein the at least one deformation sensor is a resistance based, thin film/foil based, semiconductor based, piezo based, or fiber bragg grating based strain gauge.

4. The guiding assembly for catheters according to claim 2, further comprising a first terminal connected to the at least one deformation sensor for receiving signals for strain values.

5. The guiding assembly for catheters according to claim 4, further comprising a second terminal connected to the at least one SMA actuator for providing electric current to the at least one SMA actuator.

6. The guiding assembly for catheters according to claim 1, wherein the initial shape is in a straight form, and the final shape is in a bent form or wherein the initial shape is in a bent form and the final shape is in a straight form.

7. The guiding assembly for catheters according to claim 1, further comprising a resilient member for assisting positioning the at least one SMA actuator into the initial shape from the transitional shapes or the final shape when the electric current is cut.

8. The guiding assembly for catheters according to claim 5, wherein the resilient member is in the form of a helical spring.

9. The guiding assembly for catheters according to claim 1, wherein the at least one SMA actuator and the at least one SEA member are extending longitudinal between the at least two coupling means.

10. The guiding assembly for catheters according to claim 1, further comprising a thermal insulator for thermally insulating the at least one SEA member.

11. The guiding assembly for catheters according to claim 1, further comprising a catheter tube opening for receiving a catheter.

12. The guiding assembly for catheters according to claim 7, wherein the at least two coupling means comprises recesses for mounting each of the at least one SMA actuator, each of the at least one SEA member, or the resilient member.

13. The guiding assembly for catheters according to any claim 1, further comprising a fastening means for fastening a catheter to the at least two coupling means.

14. The guiding assembly for catheters according to any claim 1, further comprising an external cover or coating surrounding the at least one SMA actuator and the at least one SEA member.

15. The guiding assembly for catheters according to claim 4, wherein the strain values are used as a feedback to a system to control the guiding assembly.

16. The guiding assembly for catheters according to claim 5, wherein the at least two coupling means comprises a SMA actuator opening and a SEA member opening adapted to respectively fit the terminals of the at least one SMA actuator and the at least one SEA member.

17. The guiding assembly for catheters according to claim 3, further comprising a first terminal connected to the at least one deformation sensor for receiving signals for strain values.

18. The guiding assembly for catheters according to claim 2, wherein the initial shape is in a straight form, and the final shape is in a bent form or wherein the initial shape is in a bent form and the final shape is in a straight form.

19. The guiding assembly for catheters according to claim 3, wherein the initial shape is in a straight form, and the final shape is in a bent form or wherein the initial shape is in a bent form and the final shape is in a straight form.

20. The guiding assembly for catheters according to claim 4, wherein the initial shape is in a straight form, and the final shape is in a bent form or wherein the initial shape is in a bent form and the final shape is in a straight form.
